(19) United States
(12) Patent Application Publication
(10) Pub. No.: US 2021/0330282 A1
(43) Pub. Date: Oct. 28, 2021

(54) BIOLOGICAL SOUND MEASUREMENT DEVICE

(71) Applicant: OMRON HEALTHCARE Co., Ltd., Kyoto (JP)

(72) Inventors: Tsuyoshi OGIHARA, Kyoto (JP); Masahiko YUMOTO, Kyoto (JP); Kosuke INOUE, Kyoto (JP); Yuki TAKUMA, Kyoto (JP)

(21) Appl. No.: 17/305,566

(22) Filed: Jul. 9, 2021

Related U.S. Application Data
(63) Continuation of application No. PCT/JP2019/049684, filed on Dec. 18, 2019.

(30) Foreign Application Priority Data
Jan. 11, 2019 (JP) 2019-003487

(51) Int. Cl. A61B 7/04; H04R 1/46

(52) U.S. Cl. CPC A61B 7/04; H04R 1/46

(57) ABSTRACT

Provided is a biological sound measurement device capable of retaining contact with the body surface in a favorable state and improving a measurement accuracy of a biological sound. A biological sound measurement device (1) includes a sound measurement unit (3) including a sound detector (33) configured to detect a biological sound, and a contact surface (30) configured to be brought into contact with the body surface (S) of a subject, a gripping portion (10) configured to be gripped by a measurer, and a coupling member (40) having a cylindrical tubular shape and elasticity and coupling the gripping portion (10) and the sound measurement unit (3). A wiring (SG) is inserted through a hollow portion of the coupling member (40).

### CROSS-REFERENCE TO RELATED APPLICATIONS

This application is the U.S. national stage application filed pursuant to 35 U.S.C. 365(c) and 120 as a continuation of International Patent Application No. PCT/JP2019/049684, filed Dec. 18, 2019, which application claims priority to Japan Patent Application No. 2019-003487, filed Jan. 11, 2019, which applications are incorporated herein by reference in their entireties.

### TECHNICAL FIELD

The present invention relates to a biological sound measurement device configured to be brought into contact with the body surface of a subject, such as an animal or a person, and measure a biological sound.

### BACKGROUND ART

There are known devices configured to utilize a microphone or the like to extract biological sounds including respiratory sounds, which are physiological sounds that originate from a flow of air generated in the respiratory tract by breathing, adventitious sounds, which are abnormal sounds generated under pathological conditions, such as wheezing or a pleural friction rub, heartbeat sounds that originate from the cardiovascular system, and the like as electrical signals (refer to, for example, Patent Documents 1 to 3).

### CITATION LIST

### Patent Literature

Patent Document 1: JP 2000-60845 A

Patent Document 2: JP 2013-123493 A

Patent Document 3: JP 2014-166241 A

### SUMMARY OF INVENTION

### Technical Problem

In order to accurately measure a biological sound, it is necessary to continually retain a contact state between a contact surface of the biological sound measurement device and the body surface of a living body in a favorable state. Patent Documents 1 to 3 do not take into consideration the problem of retaining such a contact state.

In light of the foregoing, an object of the present invention is to provide a biological sound measurement device capable of retaining contact with the body surface in a favorable state and improving a measurement accuracy of a biological sound.

### Solution to Problem

(1)

A biological sound measurement device configured to measure a biological sound of a subject including a sound measurement unit including a sound detector configured to detect the biological sound, and a contact surface configured to be brought into contact with the body surface of the subject, a gripping portion configured to be gripped by a measurer, and a coupling member having elasticity and coupling the gripping portion and the sound measurement unit.

According to (1), the gripping portion and the sound measurement unit are coupled by the coupling member having elasticity, and thus, even when the gripping portion moves with respect to the sound measurement unit in a state in which the contact surface is in contact with the body surface of the subject, this movement can be absorbed by deformation of the coupling member, thereby preventing movement of the contact surface. Accordingly, a contact state between the contact surface and the body surface can be easily continually retained, making it possible to improve a measurement accuracy of the biological sound. Further, the burden on the measurer can be reduced.

(2)

The biological sound measurement device according to (1), further including a wiring electrically connecting the sound detector and a substrate built in the gripping portion, wherein the coupling member has a structure in which the coupling member is spaced apart from the wiring and surrounding the wiring.

According to (2), the coupling member and the wiring are spaced apart, making it possible to prevent contact between the wiring and the coupling member even when the coupling member is deformed. As a result, noise can be prevented from being mixed into the sound detected by the sound detector. Further, the wiring is surrounded by the coupling member, making it possible to protect the wiring and improve designability.

(3)

The biological sound measurement device according to (2), wherein the coupling member is a member having a tubular shape.

According to (3), a force applied to the sound measurement unit from the gripping portion can be stabilized. This makes it easier to maintain the contact state between the contact surface and the body surface. Further, the wiring can be covered, making it possible to increase an air tightness and a designability of the device.

(4)

The biological sound measurement device according to (2), wherein the coupling member includes two tubular members disposed spaced apart in a direction perpendicular to the contact surface, each formed with an opening for passing the wiring, and a plurality of columnar members coupling the two tubular members and arrayed spaced apart from each other around the wiring.

According to (4), there are gaps between the plurality of columnar members, making it possible to increase a flexibility of the coupling member. Accordingly, the contact state between the contact surface and the body surface can be more easily continually retained, making it possible to improve a measurement accuracy of the biological sound.

(5)

The biological sound measurement device according to any one of (1) to (4), wherein the coupling member is positioned inward of the sound measurement unit in a state of viewing from a direction perpendicular to the contact surface.

According to (5), an object such as a finger is less likely to touch the coupling member. Thus, the occurrence of noise caused by contact between the coupling member and the object can be suppressed.

(6)

The biological sound measurement device according to any one of (1) to (5), wherein a deformation amount of the coupling member in response to a force applied in a direction parallel to the contact surface is greater than a deformation amount of the coupling member in response to a force applied in a direction perpendicular to the contact surface.

According to (6), when the contact surface of the sound measurement unit is pressed against the body surface, the deformation amount of the coupling member is small, making it possible to stably perform this task. Further, when a force is applied to the coupling member in a direction parallel to the contact surface, the deformation amount of the coupling member increases, making it possible to easily move the gripping portion in a horizontal direction while maintaining a state of contact between the contact surface and the body surface.

### Advantageous Effects of Invention

According to the present invention, it is possible to provide a biological sound measurement device capable of retaining contact with the body surface in a favorable state and improving a measurement accuracy of a biological sound.

### BRIEF DESCRIPTION OF DRAWINGS

FIG. 1 is a side view schematically illustrating an outline configuration of a biological sound measurement device 1, which is an embodiment of a biological sound measurement device according to the present invention.

FIG. 2 is a schematic view of the biological sound measurement device 1 illustrated in FIG. 1, viewed from a measurer side in a direction B.

FIG. 3 is a cross-sectional schematic view of a vicinity of a head portion of the biological sound measurement device 1 illustrated in FIG. 1.

FIG. 4 is a perspective view schematically illustrating a coupling member 40 illustrated in FIG. 1.

FIG. 5 is a diagram illustrating a configuration of a biological sound measurement device 1A which is a modified example of the biological sound measurement device 1 of FIG. 1.

FIG. 6 is a diagram illustrating the configuration of the modified example of the biological sound measurement device 1 illustrated in FIG. 1.

FIG. 7 is a perspective view schematically illustrating a coupling member 40A illustrated in FIG. 6.

FIG. 8 is a perspective view schematically illustrating a modified example of the coupling member 40A illustrated in FIG. 6.

### DESCRIPTION OF EMBODIMENTS

### Overview of Biological Sound Measurement Device of Embodiment

First, an overview of an embodiment of a biological sound measurement device according to the present invention will be described. The biological sound measurement device according to the embodiment is configured to measure, as an example of a biological sound, a pulmonary sound from a subject such as a person and, when wheezing is determined to be included in the measured sound, notify a measurer of the determination. In this way, it is possible to support the determination of the necessity of medication for the person to be measured, the determination of whether or not to take the person to the hospital, and the like.

A biological sound measurement device according to the embodiment includes a sound measurement unit including a contact surface configured to be brought into contact with the body surface of the subject such as a person, a gripping portion configured to be gripped by a measurer, and a coupling member having elasticity and coupling the gripping portion and the sound measurement unit. According to this configuration, even when a force is applied to the gripping portion in a direction parallel to the contact surface in a state in which the contact surface of the sound measurement unit is in contact with the body surface, this force can be absorbed by deformation of the coupling member, and the contact state between the contact surface and the body surface can be maintained. Accordingly, the contact state between the contact surface and the body surface can be easily retained, making it possible to improve a measurement accuracy of the biological sound.

A specific configuration example of the biological sound measurement device according to the embodiment will be described below.

### Embodiment

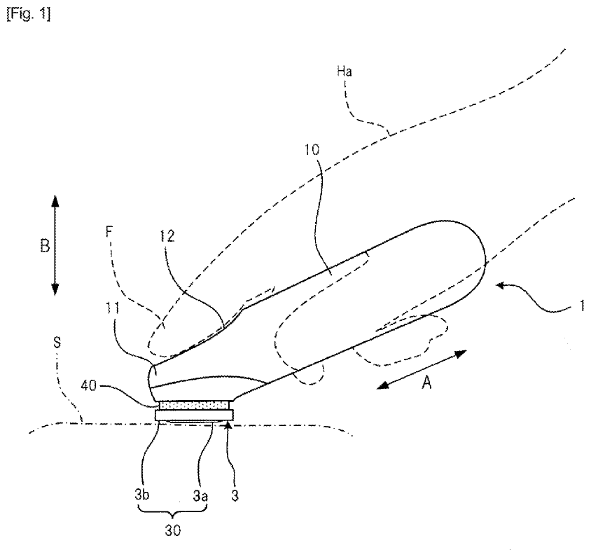

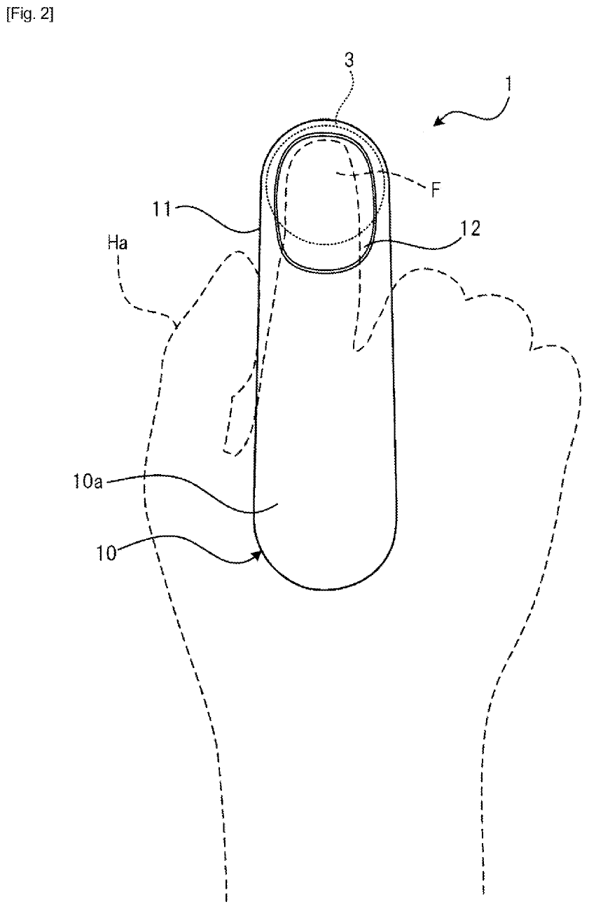

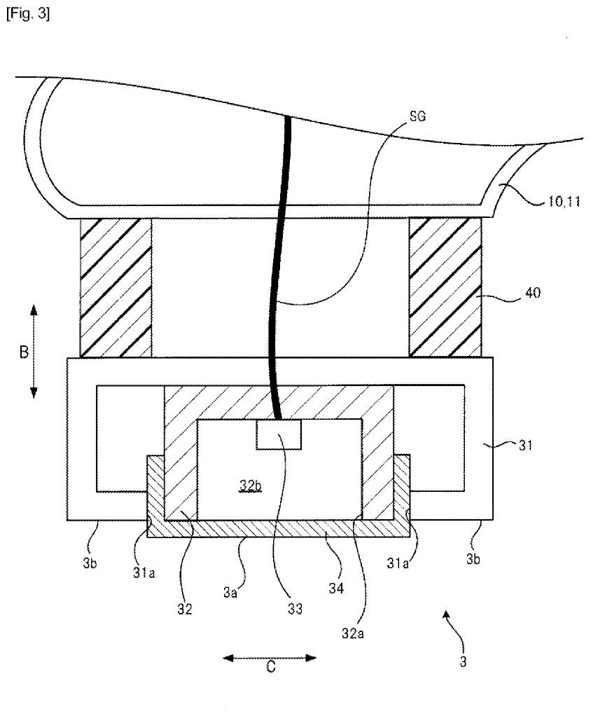

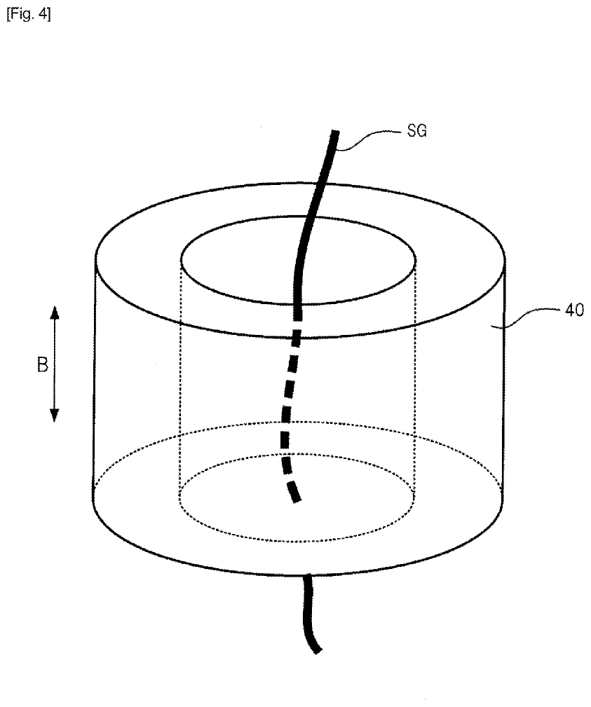

FIG. 1 is a side view schematically illustrating an outline configuration of a biological sound measurement device 1, which is an embodiment of the biological sound measurement device according to the present invention. FIG. 2 is a schematic view of the biological sound measurement device 1 illustrated in FIG. 1, viewed from the measurer side in a direction B. FIG. 3 is a cross-sectional schematic view of a vicinity of a head portion of the biological sound measurement device 1 illustrated in FIG. 1. FIG. 4 is a perspective view schematically illustrating a coupling member 40 illustrated in FIG. 1.

As illustrated in FIG. 1 and FIG. 2, the biological sound measurement device 1 includes a gripping portion 10 having a columnar shape extending in a direction A and constituted by a case of a resin, a metal, or the like. A head portion 11 is provided on one end side of this gripping portion 10. The gripping portion 10 is a portion gripped by the measurer.

A substrate (not illustrated) on which an integrated control unit configured to integrally control the entire biological sound measurement device 1 is formed, a battery (not illustrated) configured to supply a voltage required for operation, a display unit (not illustrated), and the like are provided inside the gripping portion 10.

The integrated control unit includes various processors, random access memory (RAM), read only memory (ROM), and the like, and performs a control and the like of each hardware of the biological sound measurement device 1 in accordance with a program. For example, the integrated control unit performs a process of analyzing the pulmonary sound detected by a sound detector 33 described later, and a process of notifying the measurer of an analysis result thereof.

As illustrated in FIG. 1 and FIG. 3, the head portion 11 is provided with the coupling member 40 and a sound measurement unit 3 that protrude toward one side (lower side in FIG. 1 and FIG. 3) in a direction intersecting the longitudinal direction A of the gripping portion 10. The coupling member 40 is a member coupling the head portion 11 and the sound measurement unit 3. A contact surface 30 configured to be brought into contact with the body surface S of the person to be measured is provided on a tip end of this sound measurement unit 3.

The contact surface 30 is constituted by a pressure-receiving region 3a having a circular shape, for example, and an extended region 3b having an annular shape, for example. The pressure-receiving region 3a is a flat surface required for receiving pressure from the body surface S, and the extended region 3b is a flat surface formed around the pressure-receiving region 3a and provided to increase a contact area with the body surface S. In the example of FIG. 1 and FIG. 3, the pressure-receiving region 3a protrudes slightly further toward the body surface S side than the extended region 3b, but may be formed on the same plane as the extended region 3b. The direction B illustrated in FIG. 1 is a direction perpendicular to the contact surface 30 and intersects the longitudinal direction A of the gripping portion 10.

As illustrated in FIG. 2, a state of viewing in the direction B perpendicular to the contact surface 30, a recessed portion 12 for placement of an index finger F, for example, of a hand Ha of the measurer is formed on a surface 10a of the gripping portion 10, which is opposite side to the sound measurement unit 3 side, on a portion overlapping the sound measurement unit 3.

As illustrated in FIG. 1 and FIG. 2, the biological sound measurement device 1 is used in a state in which the index finger F of the hand Ha of the measurer is placed in the recessed portion 12 of the gripping portion 10, with the contact surface 30 including the pressure-receiving region 3a of the sound measurement unit 3 being pressed against the body surface S by this index finger F.

As illustrated in FIG. 3, the sound measurement unit 3 includes the sound detector 33 such as a micro-electro-mechanical systems (MEMS) type microphone or a capacitive microphone, a housing 32 having a bottomed tubular shape, forming an accommodation space 32b accommodating the sound detector 33, and including an opening 32a, a cover 34 closing the opening 32a from outside the accommodation space 32b and forming the pressure-receiving region 3a that receives pressure from the body surface S, and a case 31 having a bottomed tubular shape and accommodating the housing 32 and the cover 34 in a state in which the cover 34 is exposed.

The housing 32 is made of a material having higher acoustic impedance than that of air and high rigidity, such as resin or metal. The housing 32 is preferably made of a material that reflects sound in a detection frequency band of the sound detector 33 in a sealed state of the housing 32 so that sound is not transmitted from the outside to the interior of the accommodation space 32b.

The cover 34 is a member having a bottomed tubular shape, and a shape of a hollow portion thereof substantially matches an outer wall shape of the housing 32. The cover 34 is made of a material having a flexibility, an acoustic impedance close to that of the human body, air, or water, and favorable biocompatibility. Examples of the material of the cover 34 include silicone and an elastomer.

The case 31 is made of resin, for example. The case 31 is formed with an opening 31a at an end portion of opposite side to the gripping portion 10 side, and a portion of the cover 34 is in a protruding and exposed state from this opening 31a. A front surface of the cover 34 exposed from this case 31 forms the pressure-receiving region 3a described above.

When the pressure-receiving region 3a is brought into close contact state with the body surface S, vibration of the body surface S generated by the pulmonary sound of the living body vibrates the cover 34. When the cover 34 vibrates, an internal pressure of the accommodation space 32b fluctuates due to this vibration and, by this internal pressure fluctuation, an electrical signal corresponding to the pulmonary sound is detected by the sound detector 33.

As illustrated in FIG. 3, the biological sound measurement device 1 includes a wiring SG for electrically connecting the sound detector 33 and the substrate described above built in the gripping portion 10. The wiring SG is drawn from the housing 32 and the case 31. The wiring SG is passed through an interior of the coupling member 40 described later and connected to the substrate in the gripping portion 10.

As illustrated in FIG. 3 and FIG. 4, the coupling member 40 is a member having a tubular shape (cylindrical tubular shape in the example illustrated in FIG. 3 and FIG. 4) and elasticity. The coupling member 40 is a member that is softer than the gripping portion 10, the case 31, and the housing 32, and is constituted by, for example, silicone, rubber, an elastomer, or resin. The coupling member 40 is preferably made of a material that does not readily generate sound when it expands and contracts. The wiring SG drawn from the case 31 is inserted through a hollow portion of the coupling member 40, and this wiring SG is drawn into the interior of the gripping portion 10 and connected to the substrate described above.

The coupling member 40 is configured such that a deformation amount (first deformation amount) in response to a force applied in the direction B is zero or negligible, and a deformation amount in response to a force applied in a direction C parallel to the contact surface 30 is sufficiently greater than the first deformation amount. Further, an inner peripheral surface of the coupling member 40 is spaced apart from the wiring SG and, even in a case in which the coupling member 40 is deformed to the maximum extent in the direction C, a size and a height in the direction B of the hollow portion of the coupling member 40 are determined to be such an extent that this inner peripheral surface and the wiring SG do not come into contact with each other.

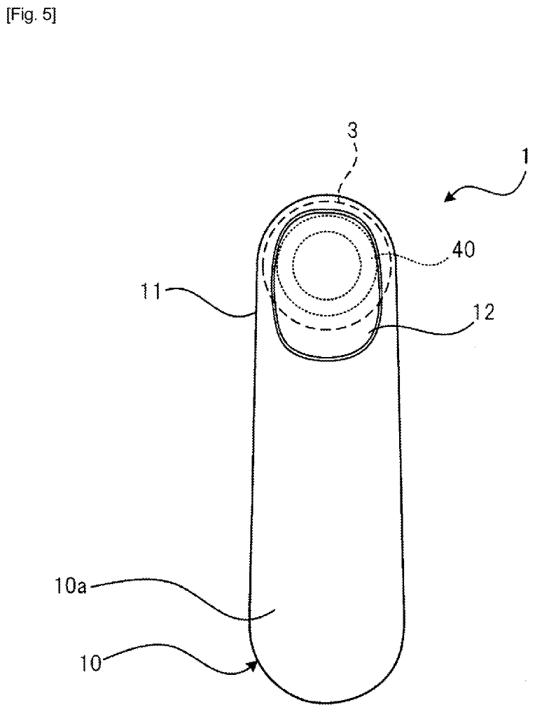

FIG. 5 is a schematic view for explaining a positional relationship between the sound measurement unit 3, the coupling member 40, and the gripping portion 10 in a state of viewing in the direction B, and is a view of the biological sound measurement device 1 of FIG. 1 viewing from the measurer side in the direction B. As illustrated in FIG. 5, in a state of viewing in the direction B, the coupling member 40 is disposed inward of the sound measurement unit 3 and is disposed inward of the gripping portion 10.

### Effects of Biological Sound Measurement Device 1

As described above, according to the biological sound measurement device 1, the gripping portion 10 and the sound measurement unit 3 are coupled by the coupling member 40 having elasticity. Thus, even when the gripping portion 10 moves with respect to the sound measurement unit 3 in the direction C in a state in which the contact surface 30 is in contact with the body surface S of the person to be measured, this movement can be absorbed by the deformation of the coupling member 40, thereby preventing movement of the contact surface 30. Accordingly, the contact state between the contact surface 30 and the body surface S can be easily continually retained, making it possible to improve the measurement accuracy of the biological sound. In particular, in a device configured to detect wheezing from a pulmonary sound, the person to be measured is presumably an infant or the like. An infant presumably moves frequently and thus, with the contact state described above being easily retainable, the burden on the measurer can be alleviated.

Further, according to the biological sound measurement device 1, the longitudinal direction (direction A) of the gripping portion 10 and the contact surface 30 intersect. Thus, in a state in which the contact surface 30 is in contact with the body surface S, the gripping portion 10 is not parallel to the body surface S. In such a configuration, the hand of the measurer is separated from the body surface S of the subject, making it possible to more remarkably achieve a retaining effect of the contact state due to deformation of the coupling member 40.

Further, according to the biological sound measurement device 1, the inner peripheral surface of the coupling member 40 and the wiring SG are spaced apart. Thus, even when the coupling member 40 is deformed, it is possible to prevent contact between the wiring SG and the coupling member 40. As a result, noise can be prevented from being mixed into the sound detected by the sound detector 33. Further, the wiring SG is surrounded by the coupling member 40, making it possible to protect the wiring SG and improve the designability of the device.

Further, according to the biological sound measurement device 1, as illustrated in FIG. 5, the coupling member 40 is disposed inward of the sound measurement unit 3, making it less likely that an object such as a finger of the measurer will touch the coupling member 40. Accordingly, the occurrence of noise caused by contact between the coupling member 40 and the object can be suppressed. Further, as illustrated in FIG. 5, the coupling member 40 is disposed inward of the gripping portion 10, making it less likely that the object such as a finger of the measurer will touch the coupling member 40 and thus the occurrence of noise can be further suppressed.

Further, in the biological sound measurement device 1, the deformation amount of the coupling member 40 in response to a force applied in the direction C parallel to the contact surface 30 is greater than the deformation amount of the coupling member 40 in response to a force applied in the direction B perpendicular to the contact surface 30. According to this configuration, when the contact surface 30 of the sound measurement unit 3 is pressed against the body surface S, the deformation amount of the coupling member 40 is small, making it possible to stably perform the pressing. Further, when a force is applied to the coupling member 40 in the direction C parallel to the contact surface 30, the deformation amount of the coupling member 40 increases. Therefore, the gripping portion 10 can be easily moved in the direction C while maintaining the state of contact between the contact surface 30 and the body surface S, making it easy to accommodate the movement or the like of the person to be measured.

### Modified Example of Biological Sound Measurement Device 1

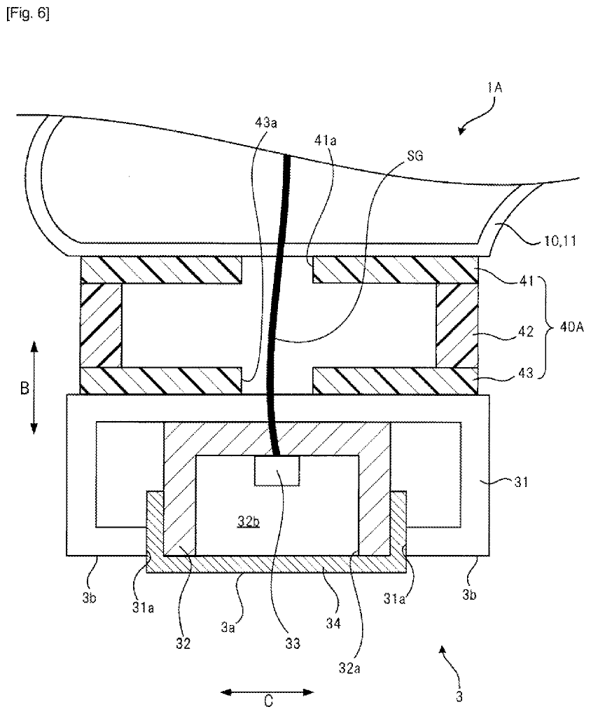

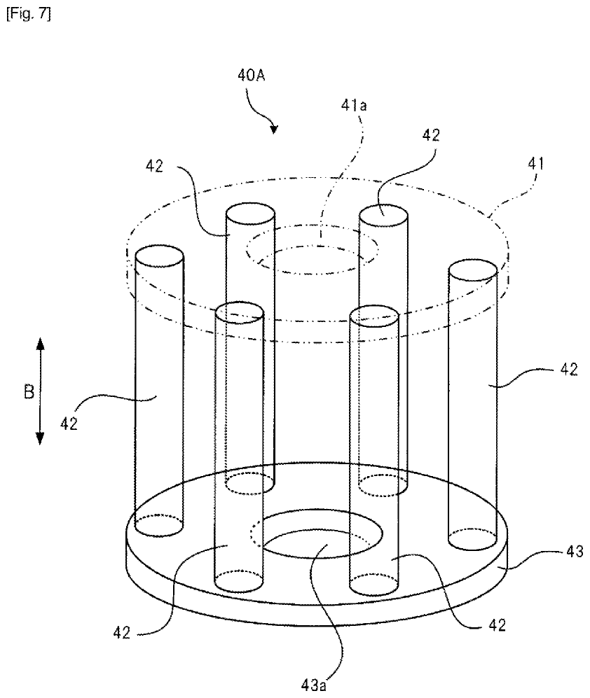

FIG. 6 is a diagram illustrating a configuration of a modified example of the biological sound measurement device 1 illustrated in FIG. 1, which corresponds to FIG. 3. A biological sound measurement device 1A illustrated in FIG. 6 has the same configuration as that of the biological sound measurement device 1 except that the coupling member 40 is changed to a coupling member 40A. FIG. 7 is a perspective view schematically illustrating the coupling member 40A illustrated in FIG. 6. In FIG. 7, a tubular member 41 described later is illustrated by a two-dot chain line for viewability of the drawing.

The coupling member 40A includes the tubular member 41, a tubular member 43, and a plurality (six in the example in FIG. 7) of columnar members 42 coupling the tubular member 41 and the tubular member 43.

The tubular member 41 is a member having a tubular shape, such as a square tubular shape or a cylindrical tubular shape, with the direction B as an axial direction and, in the example in FIG. 7, is a member having a cylindrical tubular shape. The tubular member 41 is fixed to the gripping portion 10 by an adhesive or the like.

The tubular member 43 is a member having a tubular shape, such as a square tubular shape or a cylindrical tubular shape, with the direction B as an axial direction and, in the example in FIG. 7, is a member having a cylindrical tubular shape. The tubular member 43 is disposed spaced apart from the tubular member 41 in the direction B. The case 31 of the sound measurement unit 3 is fixed to a surface of the tubular member 43 opposite to the tubular member 41 side by an adhesive or the like. In the example illustrated in FIG. 7, the tubular member 41 and the tubular member 43 have the same shape, and a center of an opening 41a of the tubular member 41 and a center of an opening 43a of the tubular member 43 coincide with each other when viewing in the direction B.

The columnar member 42 is a member having a columnar shape, such as a square columnar shape or a cylindrical columnar shape, with the direction B as an axial direction and, in the example in FIG. 7, is a member having a cylindrical columnar shape. As illustrated in FIG. 7, when viewed from the direction B, the six columnar members 42 are arrayed spaced apart from each other, surrounding each of the opening 41a of the tubular member 41 and the opening 43a of the tubular member 43.

The wiring SG of the biological sound measurement device 1A is inserted through the opening 43a of the tubular member 43 from the case 31 side. The wiring SG inserted through the opening 43a is passed through the space surrounded by the six columnar members 42 and inserted through the opening 41a of the tubular member 41. The wiring SG inserted through the opening 41a is drawn into the gripping portion 10.

Of the tubular member 41, the columnar member 42, and the tubular member 43 that constitute the coupling member 40A, at least the columnar member 42 is a member having elasticity. A deformation amount of each columnar member 42 in response to a force applied in the direction B is greater than a deformation amount of each columnar member 42 in response to a force applied in the direction C. Further, a distance from the openings 41a and 43a of the six columnar members 42 in the direction C is set to a value of such an extent that the columnar members 42 and the wiring SG do not come into contact with each other even in a state in which the six columnar members 42 are deformed to the maximum extent in the direction C. Note that the tubular member 41, the columnar member 42, and the tubular member 43 may be integrally molded, or separately molded members may be fixed to each other.

### Effects of Biological Sound Measurement Device 1A

As described above, according to the biological sound measurement device 1A, the gripping portion 10 and the sound measurement unit 3 are coupled by the coupling member 40A having elasticity and thus, even when the gripping portion 10 moves with respect to the sound measurement unit 3 in the direction C in a state in which the contact surface 30 is in contact with the body surface S of the person to be measured, this movement can be absorbed by the deformation of the six columnar members 42 of the coupling member 40A, thereby preventing movement of the contact surface 30. Accordingly, the contact state between the contact surface 30 and the body surface S can be easily continually retained, making it possible to improve the measurement accuracy of the biological sound.

Further, according to the biological sound measurement device 1A, the coupling member 40A is configured to freely deform by the six columnar members 42 arrayed spaced apart from each other, making it possible to increase a flexibility of the coupling member 40A. Accordingly, the contact state between the contact surface 30 and the body surface S can be more easily continually retained.

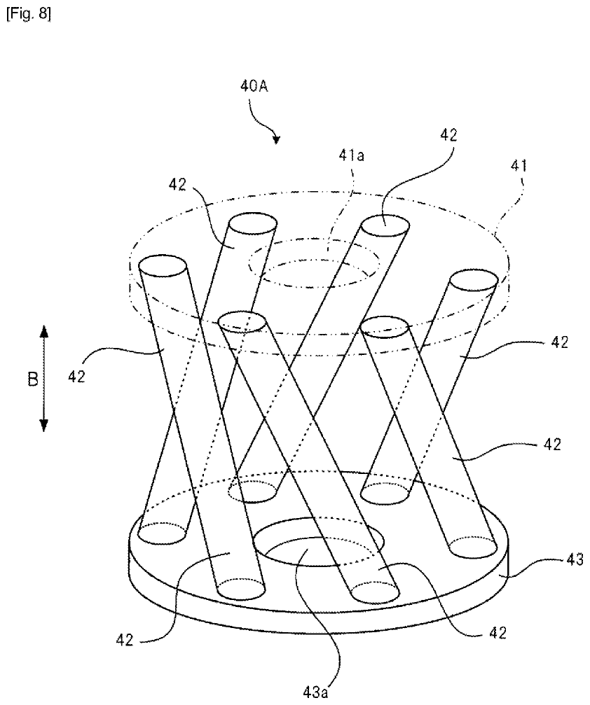

Note that the columnar member 42 of the coupling member 40A extends in the direction B in the example illustrated in FIG. 7, but may be configured to extend in a direction intersecting the direction B, as illustrated in FIG. 8. According to the configuration illustrated in FIG. 8, the flexibility of the coupling member 40A can be further increased.

While various embodiments have been described with reference to the drawings, needless to say, the present invention is not limited to such examples. It will be apparent to those skilled in the art that various changes and modifications can be made within the scope of the claims, and it is understood that these are naturally belong within the technical scope of the present invention. Further, each of the components of the above-described embodiments may be combined as desired within a range that does not depart from the spirit of the present invention.

Note that the present application is based on Japanese Patent Application filed Jan. 11, 2019 (JP 2019-003487), the contents of which are incorporated herein by reference.

### REFERENCE SIGNS LIST

1, 1A Biological sound measurement device
3 Sound measurement unit
10 Gripping portion
10a Surface
11 Head portion
12 Recessed portion
3a Pressure-receiving region
3b Extended region
30 Contact surface
31 Case
31a Opening
32 Housing
32b Accommodation space
33 Sound detector
34 Cover
40, 40A Coupling member
S Body surface
Ha Hand
F Index finger
SG Wiring

1. A biological sound measurement device configured to measure a biological sound of a subject, comprising:
a sound measurement unit including a sound detector configured to detect the biological sound, and a contact surface configured to be brought into contact with the body surface of the subject;
a gripping portion configured to be gripped by a measurer; and
a coupling member having elasticity and coupling the gripping portion and the sound measurement unit, wherein
one surface of the coupling member is connected to a surface of the sound measurement unit distanced, in a direction perpendicular to the contact surface, from a surface of the sound measurement unit on which the contact surface is disposed.

2. The biological sound measurement device according to claim 1, further comprising:
a wiring electrically connecting the sound detector and a substrate built in the gripping portion, wherein
the coupling member has a structure in which the coupling member is spaced apart from the wiring and surrounding the wiring.

3. The biological sound measurement device according to claim 2, wherein the coupling member is a member having a tubular shape.

4. The biological sound measurement device according to claim 2, wherein the coupling member includes two tubular members disposed spaced apart in a direction perpendicular to the contact surface, each formed with an opening for passing the wiring, and a plurality of columnar members coupling the two tubular members and arrayed spaced apart from each other around the wiring.

5. The biological sound measurement device according to claim 1, wherein the coupling member is positioned inward of the sound measurement unit in a state of viewing from a direction perpendicular to the contact surface.

6. The biological sound measurement device according to claim 1, wherein a deformation amount of the coupling member in response to a force applied in a direction parallel to the contact surface is greater than a deformation amount of the coupling member in response to a force applied in a direction perpendicular to the contact surface.
